(19) United States
(12) Patent Application Publication
(10) Pub. No.: US 2013/0303915 A1
(43) Pub. Date: Nov. 14, 2013

(54) ULTRASOUND APPARATUS AND METHODS TO MONITOR BODILY VESSELS

(71) Applicant: dBMEDx Inc, (US)

(72) Inventors: William L. Barnard, Maple Valley, WA (US); David B. Shine, Littleton, CO (US)

(73) Assignee: dBMEDx Inc, Littleton, CO (US)

(21) Appl. No.: 13/871,842

(22) Filed: Apr. 26, 2013

Related U.S. Application Data
(60) Provisional application No. 61/638,925, filed on Apr. 26, 2012.

(51) Int. Cl. A61B 8/08; A61B 8/00; A61B 8/02; A61B 8/13

(52) U.S. Cl. CPC A61B 8/0891; A61B 8/13; A61B 8/4236; A61B 8/02; A61B 8/461
USPC 600/449

(57) ABSTRACT

An automated 3D ultrasound abdominal vessel monitor is capable of providing automated anatomical and physiological data on the large abdominal vessels, for example the Inferior Vena Cava (IVC). The 3D ultrasound abdominal vessel monitor includes one or more ultrasound transducers built into a housing or frame that in use sits on the upper abdomen, just below the ribcage. A disposable component can serve to secure the 3D ultrasound abdominal vessel monitor to the patient and provide a coupling medium between the 3D ultrasound abdominal vessel monitor and the skin of the patient.

### BACKGROUND

1. Technical Field

This disclosure generally relates to monitoring of bodily or anatomical structures, and particularly to monitoring lumens, for instance vessels such as the inferior vena cava using ultrasound imaging.

2. Description of the Related Art

Ultrasound imaging employs transducers to produce ultrasonic pressure waves and to detect return waves in performing imaging in a variety of environments. For example, ultrasound is effectively employed in medical imaging, allowing assessments of certain bodily tissue which would not otherwise be discernible without highly invasive techniques.

There are many commercially available ultrasound systems which provide images of bodily tissue, and even flow of bodily fluids, for example blood flow.

### BRIEF SUMMARY

Disclosed is an automated three-dimensional (3D) ultrasound abdominal vessel monitor that provides automated anatomical and physiological data on the large abdominal vessels, for example the Inferior Vena Cava (IVC). The 3D ultrasound abdominal vessel monitor includes one or more ultrasound transducers built into a housing or frame that in use can be positioned on the upper abdomen, just below the ribcage, for instance proximate the xiphoid process. A disposable component can be employed to secure the 3D ultrasound abdominal vessel monitor to the patient and provide a coupling medium between the 3D ultrasound abdominal vessel monitor and the skin of the patient.

The 3D ultrasound abdominal vessel monitor may be positioned so the transducer(s) sweeps to create multiple simultaneous transverse or sagittal image planes, providing a 3D dataset from a point where the IVC meets the heart to approximately 2-8 cm inferior to that point. The data set can be collected at a rate of up to, for example, 30 frames per second for all transducers to provide real-time data on the abdominal vessels, for instance vessel volume in a selected length, across the full respiratory cycle and/or across multiple respiratory cycles.

Of specific interest is a change in volume, diameter, and/or shape of the IVC across the respiratory cycle as this is an indicator of the volume status of patient. The 2008 ACEP (American College of Emergency Physicians) Policy Statement on Emergency Ultrasound Guidelines includes the evaluation of intravascular volume status and estimation of central venous pressure (CVP) based on sonographic examination of the IVC. Changes in volume status can be reflected using sonographic evaluation of the IVC. Increased or decreased collapsibility of the vessel will help guide clinical management of the patient. The combination of the absolute diameter of the IVC and the degree of collapse with respiration allows an estimate of CVP and substitute for more invasive measurements. The 3D ultrasound abdominal vessel monitor of the present disclosure automates this recommended examination. Also see Brennan J, Blair J, Goonewardena S., Reappraisal of the Use of Inferior Vena Cava for Estimating Right Atrial Pressure, Journal of the American Society of Echocardiography, Vol. 20, Issue 7, pp.: 857-861 (Jul. 2, 2007).

There has been research on the utility of bedside ultrasound imaging machines for IVC measurements to estimate central venous pressure noninvasively and aid in assessment of the intravascular volume status of the patient. This may be of particular utility in cases of undifferentiated hypotension or other scenarios of abnormal volume states, such as sepsis, dehydration, hemorrhage, or heart failure. In addition, monitoring IVC diameter and collapse, in conjunction with BP, pericardial effusion and other clinical measures can be used to help differentiate the type of shock.

Additionally, by monitoring the IVC diameter, volume, and/or shape over time, internal blood loss can be detected from the change in the IVC maximum diameter and serial measurements can be used as a marker for response to treatment and prevention of over hydration.

The IVC is a thin-walled compliant vessel that adjusts to the body's volume status by changing its diameter depending on the total body fluid volume. The vessel contracts and expands with each respiration. Negative pressure created by the inspiration of the patient increases venous return to the heart, briefly collapsing the IVC. Exhalation decreases venous return and the IVC returns to its baseline diameter.

In states of low intravascular volume, the percentage collapse of the vessel will be proportionally higher than in intravascular volume overload states. This is quantified by the calculation of the caval index (CI-IVC):

$$CI - IVC ( \% ) = \frac{IVC exp dia - IVC insp dia}{IVC exp dia} * 100$$

where:

IVC exp dia=IVC expiratory diameter, and

IVC insp dia=IVC inspiratory diameter.

The CI-IVC is written as a percentage, where a number close to 100% is indicative of almost complete collapse (and therefore volume depletion), while a number close to 0% suggest minimal collapse (i.e., likely volume overload).

Studies have correlated the absolute IVC diameter and CI-IVC with CVP (Central Venous Pressure):

| IVC diameter | CI-IVC | CVP (cm H₂O) |
|---|---|---|
| <1.5 cm | 100% (total collapse) | 0-5 |
| 1.5-2.5 cm | >50% (partial collapse) | 6-10 |
| 1.5-2.5 cm | <50% (partial collapse) | 11-15 |
| >2.5 cm | <50% (partial collapse) | 16-20 |
| >2.5 cm | 0% (no change) | >20 |

Examples of ultrasound images of a minimal and maximal IVC are shown in FIGS. 1 and 2.

Other research (Brennan et al 2007) has correlated IVC collapse and diameter with right atrial pressure (RAP) per the table below:

### Predicted Rap Value

| | | Collapsibility Index | | |
|---|---|---|---|---|
| | | >55% | 35%-50% | <35% |
| IVC | <1.7 cm | <5 mmHg | 0-10 mmHg | indeterminate |
| SIZE | 1.7-2.1 cm | <5 mmHg | 0-10 mmHg | indeterminate |
| | >2.1 cm | 1-10 mmHg | 10-15 mmHg | 10-20 mmHg |

When a patient presents at the emergency room (ER) with shock symptoms, a central venous line is placed to assess CVP invasively. The central line is a potential site of direct infection and has the potential to increase the length of stay in the hospital. In cases where the shock was due to hypovolemia, it is possible the early and rapid introduction of IV fluids upon arrival or even in the field prior to central line placement could resolve the hypovolemia and allow the patient to avoid the placement of the central line.

In some cases, non-invasive IVC assessment is carried out with general purpose two-dimensional (2D) imaging ultrasound. This requires considerable skill and training to correctly locate and identify the IVC and adjacent anatomy. The IVC and aorta take a surprisingly tortuous path through the section of torso that needs to be imaged, which complicates the ability to locate and obtain a good image.

When attempting to measure the diameter of the vessel with a standard 2D imager it is difficult to insure the plane of the image is orthogonal to the longitudinal axis of the IVC. Furthermore, the primary axis of the ICV collapse may not be oriented orthogonal to the longitudinal axis of the ICV either. As a result, the diameter and vessel collapse as measured on the 2D image may not actually correlate well with the actual vessel geometry.

Additionally the ultrasound probe or scan head, frequently a curvilinear probe, needs to be held with just the right pressure to image the IVC without effecting the measurement. The ultrasound probe or scan head must be held on target (e.g., position and/or orientation) throughout enough respiratory cycles to obtain an accurate result. The measurement needs to be made through out the duration of the treatment as various interventions are employed; for example IV fluid replacement.

The high cost of a portable 2D imager and the extensive training required for proper use limits markets where the technology could be employed.

In some aspects, a monitor to monitor an inferior vena cava over multiple respiratory cycles includes a housing, an ultrasound system, and an output device. The ultrasound system includes an ultrasonic scan engine located at least partially in the housing and a processing subsystem communicatively coupled to the ultrasonic scan engine, which automatically detects a volume of the inferior vena cava in real time independent from heart rate. The output device is carried by the housing and is communicatively coupled to the processing subsystem to provide indications based at least in part on the detected volume of the inferior vena cava.

In some examples, the ultrasound system non-invasively detects a maximum diameter and a minimum diameter of the inferior vena cava across multiple respiratory cycles. The processing subsystem can calculate the volume of the inferior vena cava across multiple respiratory cycles. The ultrasonic scan engine can transmit a plurality of 2D ultrasound planes to form a 3D data set from which walls of the inferior vena cava are automatically detected so as to determine a size and the volume of the inferior vena cava in real time. The ultrasonic scan engine can transmit the plurality of 2D ultrasound planes in transverse sections. The ultrasonic scan engine can transmit the plurality of 2D ultrasound planes in sagittal sections.

The ultrasound system can further monitor respiration, by monitoring changes in distance from at least one local landmark within a patient over time. The at least one local landmark can be a spine of the patient. The ultrasound system can measure respiration from 1-30 times per second in real-time.

The ultrasound system can non-invasively measure a diameter of the inferior vena cava in multiple orientations around the inferior vena cava. The ultrasound system can non-invasively measure a cross-sectional area of the inferior vena cava. The ultrasound system can non-invasively measures a variation in diameter rotationally around the inferior vena cava. The processing subsystem can assess a roundness of the inferior vena cava by comparing multiple diameter measurements at different cross-sections in real time to differentiate collapse from simply reduced diameter.

The monitor can further include a self-adhering structure to facilitate positioning the housing on an abdomen of a patient without applying pressure to the abdomen relative to one or more internal organs and vessels. The self-adhering structure can include disposable adhesive pads.

The housing can include self-locating structure that conforms to a subxiphoid region. The self-locating structure can include a triangular shape which mirrors an arch formed by a base of a number of ribs and a xiphoid process of a patient.

The processing subsystem can compare a diameter of the inferior vena cava of a patient and a diameter of an aorta of the patient and calculate a ratio.

The output device can include a display, and the display presents a numerical value indicative of a relative change in diameter of the IVC. The display can further present a graphical representation of a relative change in diameter of the IVC over time. The display can present only a CI-IVC value and a heart rate value. The display can presents only a CI-IVC value, a heart rate value, and a respiration rate value.

The housing can include a substantially flat upper portion and partially cylinderical lower portion, the lower portion which is proximate a patient during use. At least a portion of at least the ultrasonic scan engine can be rotatable mounted in the lower portion of the housing. The drive subsystem can be coupled to drivingly rotate the at least portion of at least the ultrasonic scan engine about a rotational axis. The upper portion of the housing can include a pentagonal profile.

Another aspect includes a method of automatically calculating indices of a patient for clinical use. The method includes positioning a monitoring device on an abdomen of the patient, non-invasively obtaining at least one of a minimum diameter and a maximum diameter of at least one of an aorta or an inferior vena cava of the patient with the monitoring device, and automatically calculating at least one of an CI-IVC ([max IVC−min IVC]/max IVC) or an IVC/Aorta ratio based on the obtained values.

Another aspect includes a method of titrating hemodialysis. The method includes positioning a monitoring device on an abdomen of a patient, non-invasively obtaining at least one of a minimum diameter and a maximum diameter of an inferior vena cava with the monitoring device, and titrating hemodialysis based on the obtained at least one of the minimum or the maximum diameter.

Another aspect includes a method of monitoring an inferior vena cava. The method includes positioning a monitoring device on an abdomen of a patient, and scanning the inferior vena cava continuously to allow a 3D reconstruction of vessel diameter and behavior over time.

In another aspect, a monitor to monitor an inferior vena cava over multiple respiratory cycles includes a housing, an ultrasound system, and a display. The ultrasound system includes an ultrasonic scan engine located at least partially in the housing and a processing subsystem communicatively coupled to the ultrasonic scan engine, which automatically detects a volume of the inferior vena cava in real time independent from heart rate. A display is carried by the housing and commuicatively coupled to the processing subsystem to provide visual indications based at least in part on the detected volume of the inferior vena cava.

In some examples, the display presents a numerical value indicative of a relative change in diameter of the IVC. The display can present only a CI-IVC value and a heart rate value. In other examples, the display can present only a CI-IVC value, a heart rate value, and a respiration rate value. In other examples, the display can present only numerical information without any anatomical images.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a ultrasound image of a portion of an inferior vena cava at a first time during a respiratory cycle.

FIG. 2 is a ultrasound image of a portion of the inferior vena cava at a second time during a respiratory cycle, subsequent to the first time.

FIG. 3 is a front, left, bottom isometric view of a 3D ultrasound abdominal vessel monitor, according to one illustrated embodiment.

FIG. 4 is a rear, left, top isometric view of the 3D ultrasound abdominal vessel monitor, according to one illustrated embodiment.

FIG. 5 is a front, right, bottom isometric view of the 3D ultrasound abdominal vessel monitor in use positioned on a patient, according to one illustrated embodiment.

FIG. 6 is a front plan view of the 3D ultrasound abdominal vessel monitor in use positioned on the patient, according to one illustrated embodiment.

FIG. 7 is a front plan view of a display of the 3D ultrasound abdominal vessel monitor displaying CI-IVC and heart rate, according to one illustrated embodiment.

FIG. 8 is a front plan view of a display of the 3D ultrasound abdominal vessel monitor displaying CI-IVC, heart rate, and respiration rate, according to one illustrated embodiment.

FIG. 9 is a longitudinal cross-sectional view of an ultrasound scan engine according to one example.

FIG. 9A shows an ultrasound module according to one example aspect.

FIG. 10 is a transverse cross-sectional view of the ultrasound scan engine of FIG. 9.

FIGS. 10A-10C are illustrations of wobble patterns.

FIG. 11 is an example method for automatically obtaining and displaying relevant clinical indices according to one aspect.

FIG. 12 is an example method of obtaining volume information according to one example aspect.

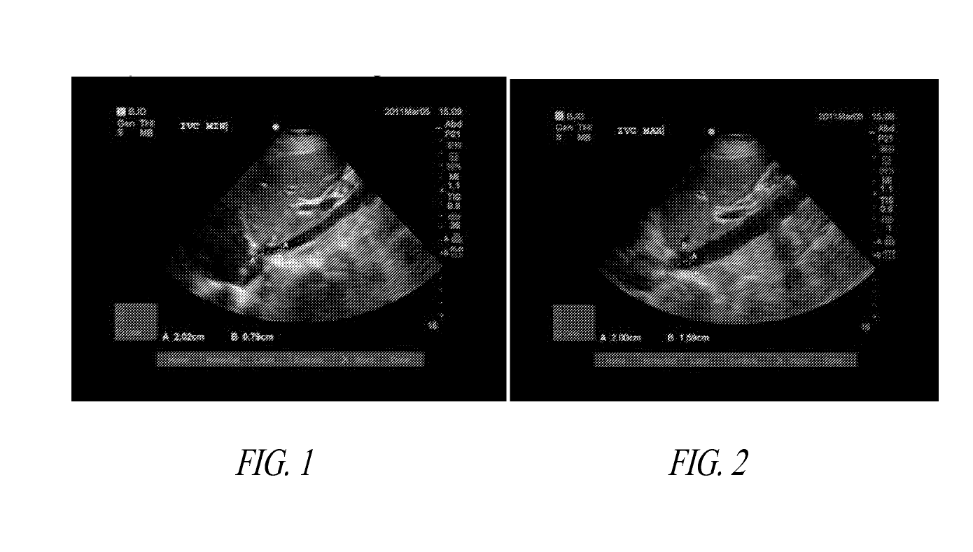

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with ultrasound systems and transducers have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

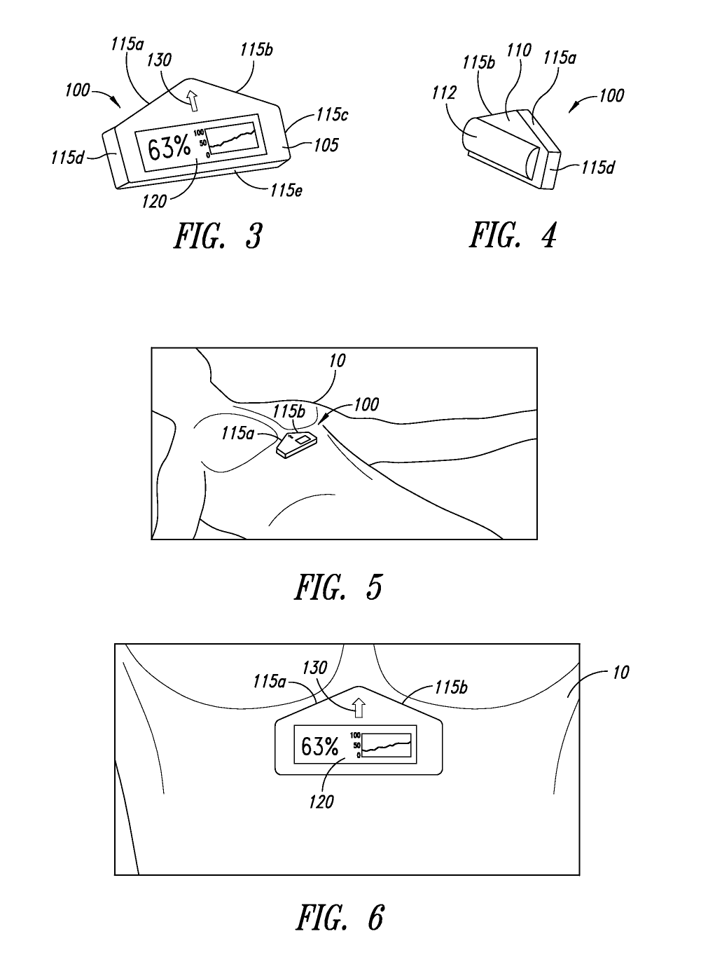

FIGS. 3 and 4 illustrate a compact, low cost, 3D ultrasound abdominal vessel monitor or device 100. The monitor 100 includes a housing 101 having a top surface 105 and an opposing back surface 110. The top surface 105 and the back surface 110 are separated by side surfaces 115a-115e, collectively referred to as side surfaces 115. The adjacent sides surfaces 115a and 115b give the monitor 100 a slightly triangular shape that aids in conforming the monitor to the subxiphoid region of a patient. The back surface 105 includes a partially cylindrical protrusion 112 that at least partially houses an ultrasound scan engine, described below. An indicia 130 on the top surface 110 provides guidance to a user for correctly orienting the monitor 100 on a patient. Although the indicia 130 is an arrow in this example, other indicia are possible. In this example, the monitor 100 includes a display area 130 that displays or visually presents the CI-IVC and optionally other parameters as well including heart rate and respiration as measured by the monitor 100.

The 3D ultrasound abdominal vessel monitor 100 can be attached to the patient's abdomen 10, as illustrated in FIGS. 5 and 6. The device 100 can be self-adhered to the abdomen using the apparatus disclosed in U.S. nonprovisional patent application Ser. No. ______, filed Apr. 26, 2013 in the names of William L. Barnard and David Bartholomew Shine and entitled “APPARATUS TO REMOVABLY SECURE AN ULTRASOUND PROBE TO TISSUE,”the contents of which are incorporated herein by reference in their entirety. In another example, disposable adhesive pads such as electrocardiograph pads, can be used to adhere the device. In either case, a suitable coupling medium may be employed.

In this example, the 3D ultrasound abdominal vessel monitor 100 is placed in the subxiphoid location at the base of the rib cage. This allows at least some of the image planes from the ultrasound scan engine to be oriented to provide a view angled under the rib cage at the lower portion of the heart where the IVC enters the right atrium. Furthermore, locating the 3D ultrasound abdominal vessel monitor 100 against the inferior part of the rib cage tends to anchor the 3D ultrasound abdominal vessel monitor 100 and allows the chest to expand and contract with respiration without placing undue pressure on the surface of the upper belly which could produce pressure on the IVC and effect the CI-IVC measurement. Such also advantageously leaves the rib cage totally unobstructed so that chest compressions and other emergency interventions can be rendered if necessary. From a privacy point of view this location is below the bra line.

In this example, the slightly triangular shape formed by the sides 115a and 115b of the 3D ultrasound abdominal vessel monitor 100 clearly indicates a position in the subxiphoid region, mirroring the arch formed by the base of the ribs and the xiphoid process. In FIGS. 5 and 6, the 3D ultrasound abdominal vessel monitor 100 is placed so that the protrusion 112 housing the ultrasound scan engine faces the patient in use, and the display 130 faces away from the surface of the patient.

As discussed in detail below, the 3D ultrasound abdominal vessel monitor 100 automatically computes the CI-IVC, CVP and other parameters in real time. The CI-IVC and/or CVP can be displayed on the display 120 of the 3D ultrasound abdominal vessel monitor. Additionally, one or more transmitters, transceivers or radios (e.g., cellular, WI-FI, Bluetooth compliant transceivers) and associated antenna(s) of the 3D ultrasound abdominal vessel monitor may wirelessly transmit the 3D image data and automatically computed numerical data (such as the CI-IVC) remotely to a receiving station such as a patient monitoring system, which for example may be in a same room as the 3D ultrasound abdominal vessel monitor.

In the example in FIGS. 3-6, the display 120 visually present the CI-IVC and optionally other parameters as well including heart rate and respiration as measured from the image data. Obtaining the heart rate from the beating heart itself is a more robust method to determine heart rate than trying to locate the pulse in extremity vasculature—either with a stethoscope or blood pressure cuff/sphygmomanometer or pulse oximeter. There are numerous situations (i.e. shock or trauma) that will degrade or prevent the measurement of pulse at the extremity.

The display 120 can be an LCD screen or other suitable display. The display 120 may take the form of touch screen display, positioned on or recessed in, or slightly protruding from a surface of a housing of the 3D ultrasound abdominal vessel monitor. The display 120 shows relevant parameters, including a calculation of the relative change in diameter of the IVC. The display 120 could also present a graphical representation of the relative change over time or other parameters over time, as shown in FIG. 3. This information could also be wirelessly transmitted to a receiver such as a base station or mobile device (phone, tablet, computer) for storage or remote monitoring.

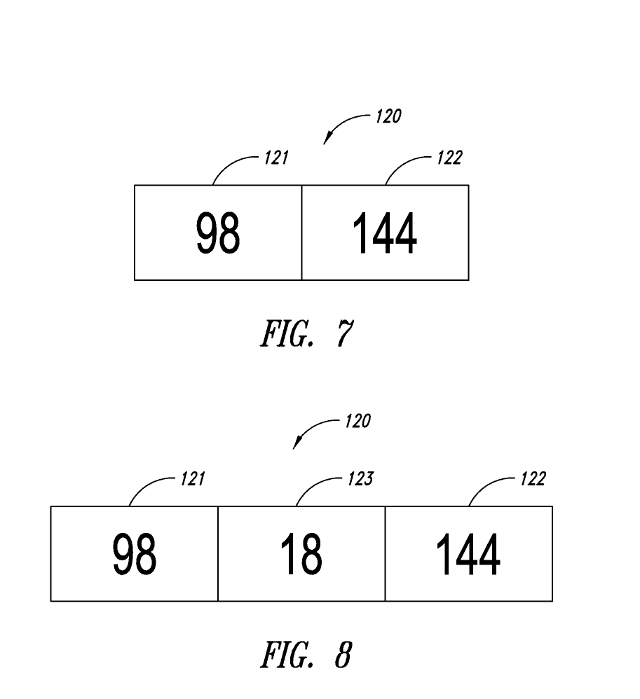

FIGS. 7 and 8 illustrate possible values displayed on the vessel monitor display 120. In FIG. 7, the display 120 of the 3D ultrasound abdominal vessel monitor 100 displays a CI-IVC value and heart rate value. For example, the display of the 3D ultrasound abdominal vessel monitor may display the CI-IVC value in a display portion 121 located on one side (e.g., left side) of a patient's midline using a first color (e.g., blue) and the heart rate value in a display portion 122 located on the other side (e.g., right side) of the patient's midline using a second color (e.g., red). In this example, the right/left orientation of the numeric displays is visually aligned with the actual patient anatomy to also provide an indication to the operator, as does the color selection, as to the meaning of each number.

In another example, illustrated in FIG. 8, the display 120 of the 3D ultrasound abdominal vessel monitor 100 displays may additionally display a respiration rate. The 3D ultrasound abdominal vessel monitor 100 can detect the anterior and posterior rib cage as well as the spine, which thereby allows measurement of the relative expansion of the rib cage as a surrogate for respiration rate. This value is typically computed as breaths per minute. In this example, the display 120 of the 3D ultrasound abdominal vessel monitor 100 displays the CI-IVC in the display portion 121 located on one side (e.g., left side) of a patient's midline using a first color (e.g., blue), the heart rate in the display portion 122 located on the other side (e.g., right side) of the patient's midline using a second color (e.g., red), and the respiration rate in a display portion 123 located between (e.g., on the patient's midline) using a third color (e.g., green or amber).

The collapse of the IVC may also vary depending on the type of breathing, specifically breathing due largely to diaphragm movement versus breathing due largely to chest expansion. Due to the wide field of view of the monitor, it will be able to monitor both diaphragm movement and rib cage expansion and determine which is the dominant force and alert the user to increase the utility of the IVC geometry data.

Other display options include graphically showing the IVC, heart and lungs as icons or other visual representations to indicate the meaning of each digital number.

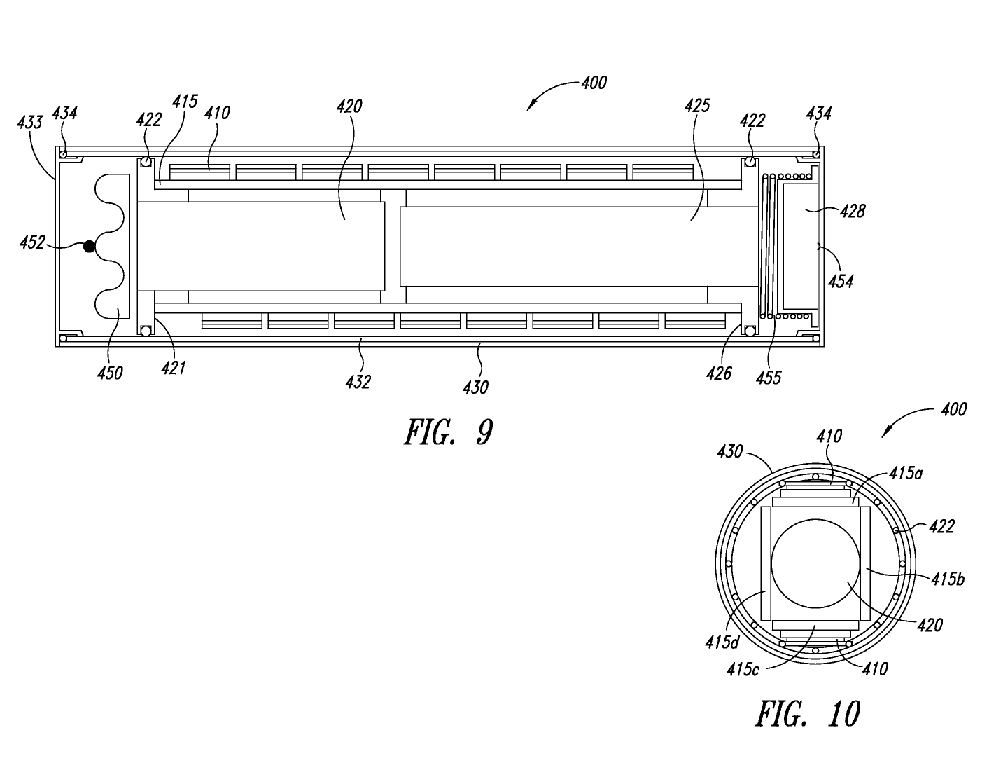

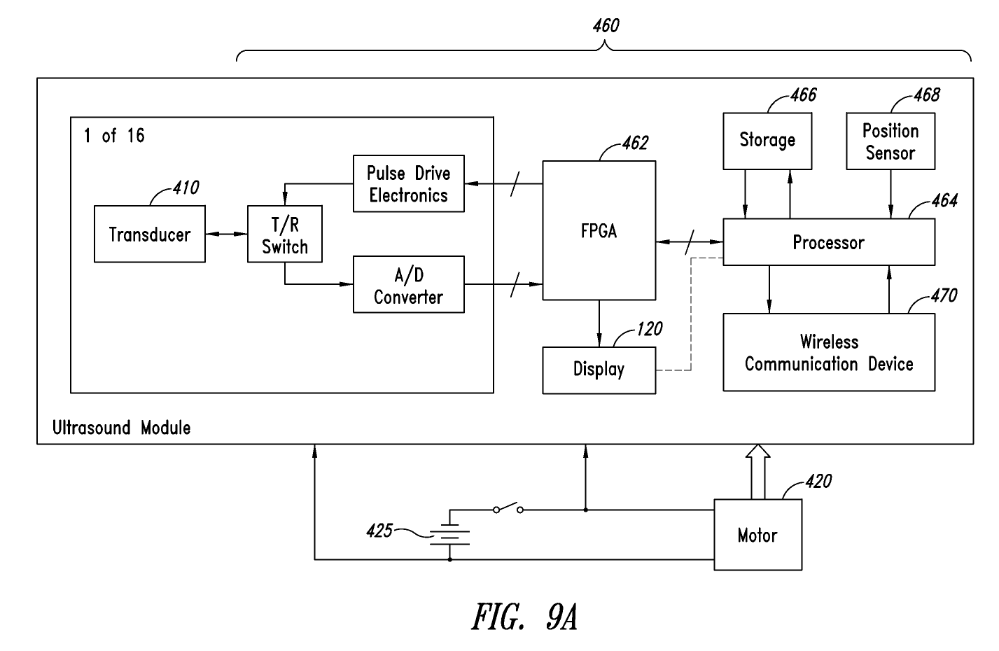

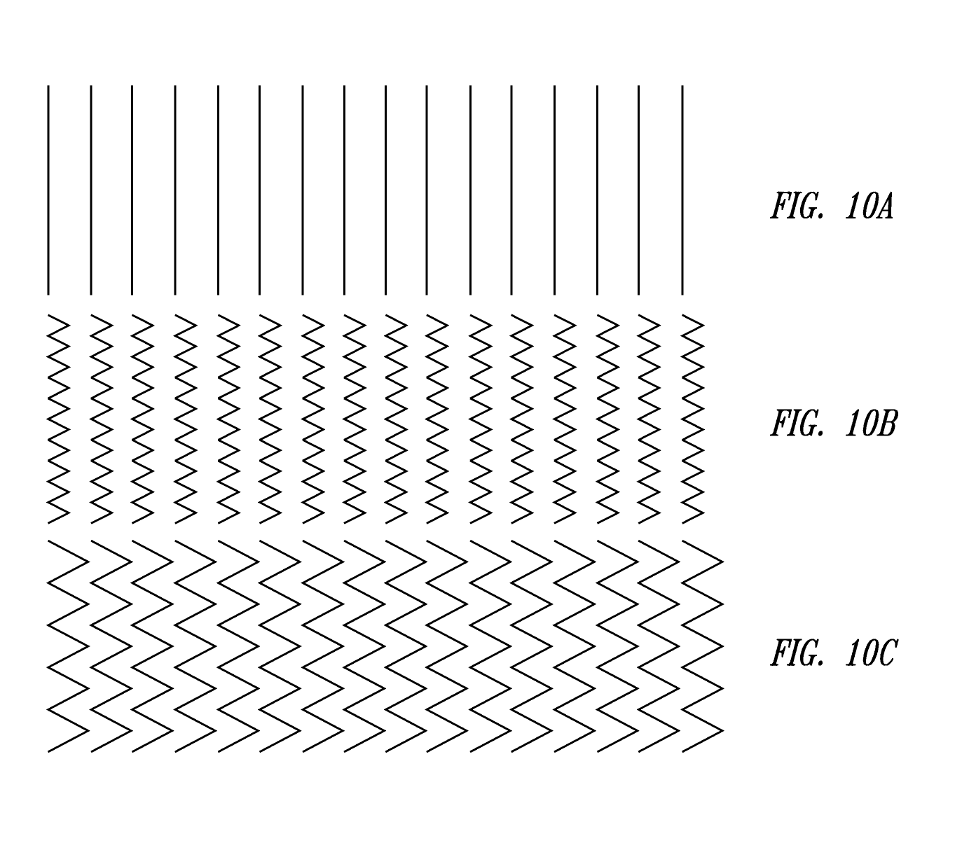

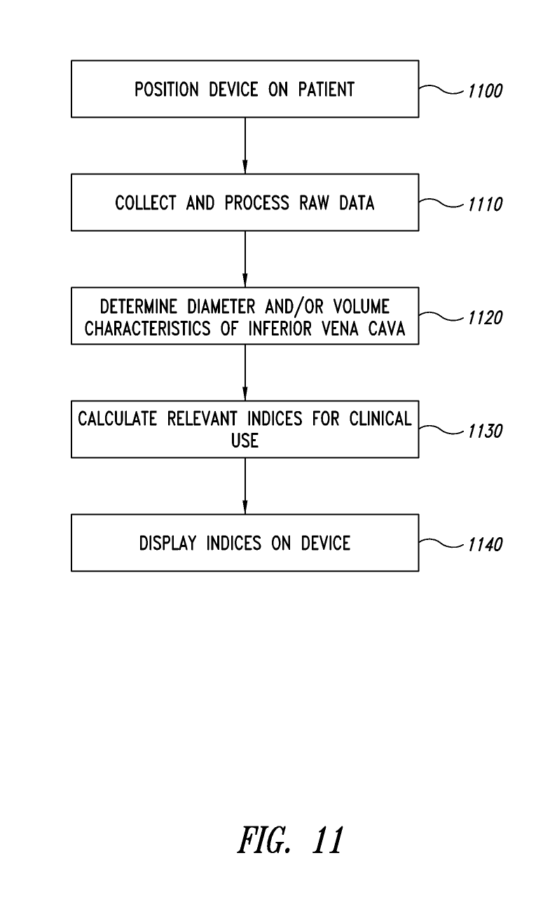

The 3D ultrasound abdominal vessel monitor can utilize one or more transducers, swept mechanically or electronically to create the desired scan planes. FIGS. 9 and 10 illustrate one example of an ultrasound scan engine that can be used in the 3D ultrasound abdominal vessel monitor 100.

In this example, an ultrasound scan engine 400 includes a motor 420 and battery 425 are located in the center of a spinning apparatus. The apparatus includes a static shaft 452, a sphere bushing 454, and a ferrite pot core 428. Transducers 410 and the associated electronics are located on printed circuit boards 415a-415d that collectively form a box 415 around the motor 420 and battery 425. In the illustrated example, half of the transducers 410 are located on one side of the box 415 and the other half are on the opposite side. As the entire assembly rotates each bank of transducer comes to the front and ultrasound is fired. This mechanism inherently balances the weight of the transducers 410 to facilitate smooth, low power, low friction spinning. It also allows us to use a wider aperture transducer and still achieve a tight lateral spacing. In this example, 10 mm aperture transducers are used to get high power which is focused deeper into the chest cavity. However, this architecture allows the transducers to be interleaved resulting in 6 mm of spacing. In another example, all four sides of the PCB “box” 415 include transducers 415, creating even tighter spacing and increased resolution.

The PCB box 415 has connections across all four corners via soldered half-vias; these are normal vias that have been cut such that only half the cylindrical via is left exposed on the very edge of the PCB. This makes a very stiff structure and is all we need to span the distance between our bearing surfaces.

To create a robust and mechanically rigid assembly, a thin wall tube 430 reinforced with a stainless steel sleeve 432 is used to provide a support structure for the static rod 452 and the outer surface for the ball bearings 422. The ball bearings 422 are supported by a motor hub 421 and a battery hub 426. In one example, the stainless steel shell 432 has a large opening where the ultrasound exits through an LDPE or HDPE window. In another example, the thickness of the LDPE/HDPE acoustic window is increased to eliminate the stainless steel sleeve 432. Other bearing solutions are possible, including hydrostatic bearings and simple lubricious plastic rub bearings. Snap-lock end caps 433 and O-rings 434 create a sealed environment that can be filled with, for example, a suitable non-corrosive, bio-compatible coupling fluid.

In the present example, quality segmentation or automatic recognition of an arterial or venous vessel is facilitated by obtaining a sufficient resolution of the ultrasonic data. The lumen of the major trunk vessels in the human abdominal region can be as small as 12 mm across in a smaller framed adult female. The vessels also follow relatively torturous paths which can complicate segmentation unless a large 3D field of view with high resolution is employed.

The ultrasound scan engine described above includes 16 transducers spaced that are 6 mm apart and that get swept through a full 360° arc, creating a very wide field of view. In particular the unusually large arc of the biologically relevant portion of the field of view (180°) allows 3D ultrasound abdominal vessel monitor 100 to look up under the rib cage to see the aorta exiting the heart. This provides the large 3D field of view.

In order to increase the spatial resolution of the ultrasound data a mechanical “wobble” motion is added by way of the wobble wheel 452 and the compression spring 455 so that the transducers 410 sweep back and forth several times as they simultaneous rotate around the main axis. This dramatically increases spatial resolution while still using a single uni-directional spinning motor. Example wiggle patterns are illustrated in FIGS. 10A-10C. FIG. 10A illustrates the pattern that would result from no wiggle. FIG. 10B illustrates a 3 mm wiggle in combination with transducers that are spaced 6 mm apart. FIG. 10C illustrates a 6 mm wiggle in combination with transducers that are spaced 6 mm apart.

FIG. 9A shows an ultrasound module which is rotated within the thin wall tube 430 by the motor 420 and powered by the battery 425 according to one illustrated embodiment. In particular, the illustrated example of FIG. 9A includes a control and processing system 460 with various electrical components that enable functionality of the ultrasound probe ultrasound scan engine 400. For example, one or more application specific integrated circuits (ASICs) programmable gate or arrays (PGAs) 462 may be coupled to a microprocessor 464 for controlling and coordinating the various functions of the ultrasound scan engine 400, including rotation of the transducers 410 and PCB box 415 and transmitting and receiving of high frequency sound waves from each of the transducers 410. The control and processing system 460 may include discrete analog to digital converters (ADCs) and/or discrete digital to analog converters (DACs). Alternatively, the ADC and/or DAC functions may be implemented in the ASIC or PGA. The control and processing system 460 may further include power supply circuitry, for example an inverter, rectifier, step up or step down converter, transformer, etc. The control and processing system 460 may further include transmit and timing control circuitry to control waveform timing, aperture and focusing of the ultrasound pressure waves.

The control and processing system 460 further includes a storage device 466 (e.g., serial flash), a rotational position sensor 468 (e.g., hall-effect sensor, optical encoder) and a wireless communication device 470 (e.g., Bluetooth radio module or other suitable short-range wireless device). The storage device 466 enables temporary storage of data, control signals, instructions and the like. The position sensor 468 enables the control and processing system 460 to coordinate the transmitting and receiving of high frequency sound waves from each of the transducers 410 with the rotational position of the ultrasound scan engine 400. The wireless communication device 470 enables data output from the ultrasound scan engine 400 to remote devices for further processing or evaluation, such as, for example, a remote evaluation device having components such as a monitor or other display devices, a keyboard, a printer and/or other input and output devices. In this manner, diagnostic data may be collected with the ultrasound scan engine 400 in a particularly small form factor of package, such that the user may obtain such data with minimal bother or inconvenience to the host of the target sample and without interference from otherwise bulky components or cables. Of course, in some embodiments an extensive user interface, including for example a display, keypad, printer and/or other input and output devices may be integrated with ultrasound scan engine 400 for further evaluation or processing onboard. The control and processing system 460 may further include or be communicatively coupled to the display 120.

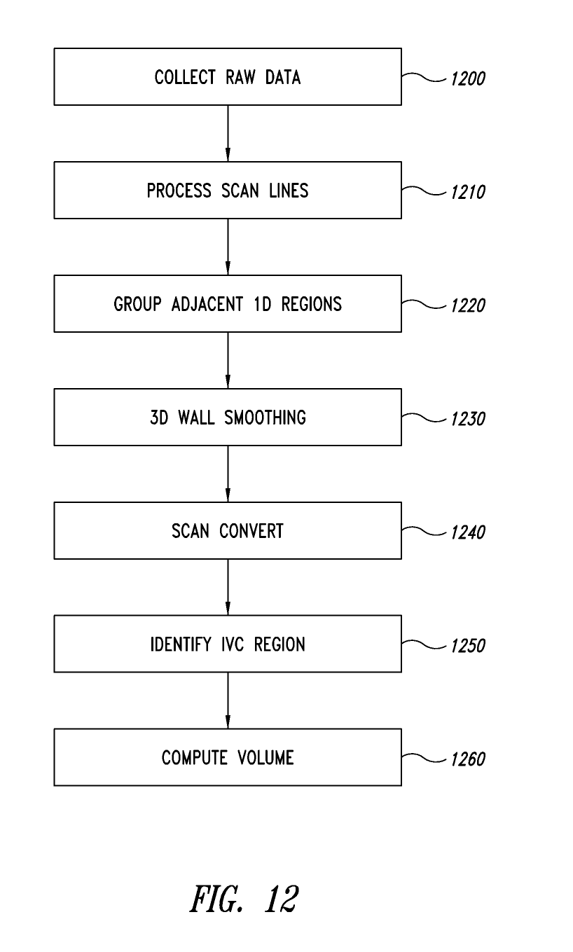

FIG. 11 provides an overview of one example method according to the present disclosure. Initially the 3D ultrasound abdominal vessel monitor 100 is positioned on the abdomen of the patient at 1100. The ultrasound scan engine 400 then collects and processes raw data at 1110. The processing system 460 then determines the diameter volume, diameter, and/or shape of the IVC across the respiratory cycle at 1120. The relevant indices for clinical use, including, for example, the CI-IVC value, heart rate value, and respiration rate, are then calculated at 1130. These indices can then be displayed on the display 120 as described above.

The 3D ultrasound abdominal vessel monitor 100 may be used to improve emergency medicine in the field. So for instance, the 3D ultrasound abdominal vessel monitor 100 is simple enough and robust enough to use in an emergency aid van or ambulance. An emergency medical technician (EMT) can place the 3D ultrasound abdominal vessel monitor on the patient either in the field or en route to the hospital. The technician could make a phone call to an attending emergency physician and relay the stats being provided by the 3D ultrasound abdominal vessel monitor. One common intervention is starting an IV to replace fluid volume and this could started as early as possible with knowledge of a collapsing vena cava. In this role the 3D ultrasound abdominal vessel monitor may include a microphone to record any verbal notes the technician wanted to make, such as when and how much IV fluid was added to the patient. The 3D ultrasound abdominal vessel monitor may include nontransitory non-volatile memory (e.g., FLASH, EEPROM) that records the 3D segmented anatomy, computed statistics, compressed full motion video, and/or the voice recording. Upon entrance to the urgent care or emergency care room this information could be requested and transmitted over a wireless link to a base station, computer, tablet or other mobile device. Some field situations such as cardiac tamponade may benefit from the tablet or other mobile display device that would allow for a diagnosis in the field where a 3D image of the heart and the pericardial sac around the heart may be displayed; in this case the intervention of aspirating the pericardial sac can be life-saving.

The 3D ultrasound abdominal vessel monitor 100 could also be used by a general practitioner to monitor IVC parameters over time (weekly, every office visit) for patients at risk for heart failure as IVC collapse can be used as an indicator of elevated right atrium pressure.

In patients undergoing hemodialysis, automated IVC monitoring can be used to maintain proper volume status and prevent hypovolemia. This improves outcomes and quality of life and reduces adverse events.

FIG. 12 illustrates an example method for obtaining the relevant volume information with the 3D ultrasound abdominal vessel monitor 100. The device 100 begins by collecting raw data with the ultrasound scan engine 400 at 1200. The monitor 100 then processes the pulse-echo ultrasound using standard amplitude imaging and color flow Doppler techniques. The color flow Doppler is a standard technique known to those skilled in the art to identify the presence and direction of blood flow.

After collecting the 3D raw data for the entire volumetric field of view the scan lines are processed at 1210. A standard one dimensional Sobel filter is run along each scan line. The Sobel filter identifies “edges” or large first derivatives in the data. In this example, the image processing is performed along each cylindrical coordinate scan line, as opposed to a Cartesian coordinate alternative, because as the ultrasound passes through the body it gets differentially attenuated by different tissue and anatomy. By performing image processing along each cylindrical coordinate scan line, one can properly understand the echo from a structure by taking into account what happened to the pulse proximal to that particular echo. In addition to the edge data, the absolute level of return and the color Doppler value is calculated for each voxel (volume pixel) in the scan line. This data is combined to identify linear regions of potential vessels. A negative slope followed by an anechoic section with Doppler flow return followed by a positive slope would be a potential vessel region. A front wall is identified by the negative slope location and a back wall is identified by the positive slope.

After each scan line is processed into potential regions with a front wall and a back wall, the individual linear regions are analyzed to see if there are adjacent regions identified in adjacent scan lines at 1220. This enables the creation of 3D regions that are potential vessels. This processing can be done in the original cylindrical coordinate system to avoid the processing expense of scan conversion to Cartesian coordinates in areas that are not viable 3D vessels regions.

The region wall locations are then run through a standard smoothing algorithm at 1230 using the input wall locations as a starting point in the raw data to adjust and precisely locate the wall locations based on correlation/smoothing in 3D.

Then the wall locations are then scan converted to 3D Cartesian coordinates at 1240. Simple heuristics are then employed at 1250 to complete the segmentation of the inferior vena cava and the descending aorta. For instance, the two vessels are typically next to each other and have flow in opposite directions. The aorta is the vessel attached to the lower part of the heart visible to our extreme field of view. The vessels can be tracked over time and it is expected that the identified aorta will have dimensional changes with a cardiac cycle frequency (50-120 beats/min) while the IVC will have dimensional changes in sync with respiration (10-30 breaths/min).

The volume of the vessel is then calculated at 1260 by integrating and counting the number of Cartesian coordinate voxels inside the vessel region. Since the vessel is not fully contained with even the enlarged field of view that is possible with the monitor 100, it is possible to arbitrarily choose a defined length to integrate across and maintain that length and relative location in the field of view from one frame to the next. In one example, the length is 10 cm which is computed along the length of the vessel no matter how torturous the path taken by the vessel.

The methods illustrated and described herein may include additional acts and/or may omit some acts. The methods illustrated and described herein may perform the acts in a different order. Some of the acts may be performed sequentially, while some acts may be performed concurrently with other acts. Some acts may be merged into a single act through the use of appropriate circuitry.

The various embodiments described above can be combined to provide further embodiments.

To the extent that they are not inconsistent with the teachings herein, the teachings of: U.S. patent application Ser. No. 12/948,622, filed Nov. 17, 2010; U.S. provisional patent application Ser. No. 61/573,493, filed Sep. 6, 2011; and U.S. provisional patent application Ser. No. 61/621,877, filed Apr. 9, 2012; U.S. provisional patent application Ser. No. 61/638,833, filed Apr. 26, 2012; and U.S. provisional patent application Ser. No. 61/638,925, filed Apr. 26, 2012; and U.S. nonprovisional patent application Ser. No. ______, filed Apr. 26, 2013 in the names of William L. Barnard and David Bartholomew Shine and entitled “APPARATUS TO REMOVABLY SECURE AN ULTRASOUND PROBE TO TISSUE” are each incorporated herein by reference in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A monitor to monitor an inferior vena cava over multiple respiratory cycles, the device comprising:
a housing;
an ultrasound system that includes an ultrasonic scan engine located at least partially in the housing and a processing subsystem communicatively coupled to the ultrasonic scan engine, which automatically detects a volume of the inferior vena cava in real time independent from heart rate; and
an output device carried by the housing and communicatively coupled to the processing subsystem to provide indications based at least in part on the detected volume of the inferior vena cava.

2. The monitor of claim 1 wherein the ultrasound system non-invasively detects a maximum diameter and a minimum diameter of the inferior vena cava across multiple respiratory cycles.

3. The monitor of claim 1 wherein the processing subsystem calculates the volume of the inferior vena cava across multiple respiratory cycles.

4. The monitor of claim 1 wherein the ultrasonic scan engine transmits a plurality of 2D ultrasound planes to form a 3D data set from which walls of the inferior vena cava are automatically detected so as to determine a size and the volume of the inferior vena cava in real time.

5. The monitor of claim 4 wherein the ultrasonic scan engine transmits the plurality of 2D ultrasound planes in transverse sections.

6. The monitor of claim 4 wherein the ultrasonic scan engine transmits the plurality of 2D ultrasound planes in sagittal sections.

7. The monitor of to claim 2 wherein the ultrasound system further monitors respiration, by monitoring changes in distance from at least one local landmark within a patient over time.

8. The monitor of claim 7 wherein the at least one local landmark is a spine of the patient.

9. The monitor of claim 7 wherein the ultrasound system measures respiration from 1-30 times per second in real-time.

10. The monitor of claim 1 wherein the ultrasound system non-invasively measures a diameter of the inferior vena cava in multiple orientations around the inferior vena cava.

11. The monitor of claim 1 wherein the ultrasound system non-invasively measures a cross-sectional area of the inferior vena cava.

12. The monitor of claim 1 wherein the ultrasound system non-invasively measures a variation in diameter rotationally around the inferior vena cava.

13. The monitor of claim 1 wherein the processing subsystem assesses a roundness of the inferior vena cava by comparing multiple diameter measurements at different cross-sections in real time to differentiate collapse from simply reduced diameter.

14. The monitor of claim 1, further comprising:
a self-adhering structure to facilitate positioning the housing on an abdomen of a patient without applying pressure to the abdomen relative to one or more internal organs and vessels.

15. The monitor of claim 1 wherein the self-adhering structure can include disposable adhesive pads.

16. The monitor of claim 1 wherein the housing includes self-locating structure that conforms to a subxiphoid region.

17. The monitor of claim 16 wherein the self-locating structure includes a triangular shape which mirrors an arch formed by a base of a number of ribs and a xiphoid process of a patient.

18. The monitor of claim 1 wherein the processing subsystem compares a diameter of the inferior vena cava of a patient and a diameter of an aorta of the patient and calculates a ratio.

19. The monitor of claim 1 wherein the output device comprises a display, and the display presents a numerical value indicative of a relative change in diameter of the IVC.

20. The monitor of claim 19 wherein the display further presents a graphical representation of a relative change in diameter of the IVC over time.

21. The monitor of claim 1 wherein the output device comprises a display, and the display presents only a CI-IVC value and a heart rate value.

22. The monitor of claim 1 wherein the output device comprises a display, the display presents only a CI-IVC value, a heart rate value, and a respiration rate value.

23. The monitor of claim 1 wherein the housing includes a substantially flat upper portion and partially cylinderical lower portion, the lower portion which is proximate a patient during use.

24. The monitor of claim 23 wherein at least a portion of at least the ultrasonic scan engine is rotatable mounted in the lower portion of the housing, and further comprising:
a drive subsystem coupled to drivingly rotate the at least portion of at least the ultrasonic scan engine about a rotational axis.

25. The monitor of claim 23 wherein the upper portion of the housing has a pentagonal profile.

26. A method of automatically calculating indices of a patient for clinical use, comprising:
positioning a monitoring device on an abdomen of the patient;
non-invasively obtaining at least one of a minimum diameter and a maximum diameter of at least one of an aorta or an inferior vena cava of the patient with the monitoring device; and
automatically calculating at least one of an CI-IVC ([max IVC−min IVC]/max IVC) or an IVC/Aorta ratio based on the obtained values.

27. A method of titrating hemodialysis, comprising:
positioning a monitoring device on an abdomen of a patient;
non-invasively obtaining at least one of a minimum diameter and a maximum diameter of an inferior vena cava with the monitoring device; and
titrating hemodialysis based on the obtained at least one of the minimum or the maximum diameter.

28. A method of monitoring an inferior vena cava, comprising:
positioning a monitoring device on an abdomen of a patient; and
scanning the inferior vena cava continuously to allow a 3D reconstruction of vessel diameter and behavior over time.

29. A monitor to monitor an inferior vena cava over multiple respiratory cycles, the device comprising:
a housing;
an ultrasound system that includes an ultrasonic scan engine located at least partially in the housing and a processing subsystem communicatively coupled to the ultrasonic scan engine, which automatically detects a volume of the inferior vena cava in real time independent from heart rate; and
a display carried by the housing and commuicatively coupled to the processing subsystem to provide visual indications based at least in part on the detected volume of the inferior vena cava.

30. The monitor of claim 29 wherein the display presents a numerical value indicative of a relative change in diameter of the IVC.

31. The monitor of claim 29 wherein the display presents only a CI-IVC value and a heart rate value.

32. The monitor of claim 29 wherein the display presents only a CI-IVC value, a heart rate value, and a respiration rate value.

33. The monitor of claim 29 wherein the display presents only numerical information without any anatomical images.
